(19) United States
(12) Patent Application Publication
(10) Pub. No.: US 2013/0289300 A1
(43) Pub. Date: Oct. 31, 2013

(54) METHOD AND SYSTEM FOR SEPARATING LINALYL ACETATE FROM LAVENDER ESSENTIAL OIL AND PREPARING ITS DERIVATIVES

(71) Applicants: Zer-Ran Yu, Chiayi City (TW); Be-Jen Wang, Chiayi City (TW)

(72) Inventors: Zer-Ran Yu, Chiayi City (TW); Be-Jen Wang, Chiayi City (TW)

(21) Appl. No.: 13/455,356

(22) Filed: Apr. 25, 2012

(51) Int. Cl. C07C 67/58

(52) U.S. Cl. 560/183

(57) ABSTRACT

The present invention provides a method and system for separating linalyl acetate from Lavender essential oil and preparing its derivatives, whereby the supercritical fluid technology is used to feed Lavender essential oil and supercritical solvent into the first separating tank, where linalyl acetate and linalool are separated from Lavender essential oil; then linalyl acetate is conveyed to the second separating tank, where linalyl acetate is separated with wax-containing oily substance to obtain high-concentration linalyl acetate; next linalool and vitamin C solution are conveyed to a reaction tank for esterification synthesis, and the reaction tank is filled with acid resin catalyst to obtain linalool-vitamin C derivative; with this design, it is possible to reduce the nervous tension, and adjust high-concentration linalyl acetate affecting physiological and psychological stress, as well as vitamin C derivative with anti-oxidizing oily fragrance.

### BACKGROUND OF INVENTION

1. Field of the Invention

The present invention relates generally to Lavender essential oil technology, and more particularly to an innovative one which involves the method and system for separating linalyl acetate from Lavender essential oil and preparing its derivatives for various purposes.

2. Description of Related Art

Lavender essential oil is obtained from the flower and stem of Lavender, of which the higher proportion of ester means better quality and higher price. The essential oil is a colorless to yellowish liquid presenting sweet taste, flower fragrance and pleasant feeling. True Lavender extract contains linalool (30-40%), linalyl acetate (46-54%) and camphor (0.5-0.9%). The linalool and linalyl acetate could make users feel comfortable, reduce the nervous tension, adjust physiological and psychological stress caused by sympathetic and parasympathetic nerve disorder, and alleviate the anxiety.

Recently, the prevailing essential oil -contained aromatherapy is used to adjust the physical and mental state via different proportions of extracts, for example, by permitting inhalation through 1-2% linalool or linalyl acetate with sedative effect or relaxation and sleep-inducing effect of perfumes and aromatherapy. Lavender essential oil has sedation and refreshing effect, depending on the human physique and acceptability. For those prone to nervous anxiety, sedative extract, e.g.: Lavender essential oil containing high linalyl acetate, shall be used; to the contrary, for those prone to lassitude and fatigue, refreshing extract, e.g.: Lavender essential oil containing high linalool, shall be used.

Lavender essential oil is generally prepared by steam distillation or solvent extraction method, of which steam distillation likely causes decomposition of some linalyl acetate, and solvent extraction is used in a manner to make the concrete product from distilled solvent contain waxes with numerous paraffins, which will be diluted by numerous ethanol, and then cooled down to remove centrifugally waxes and obtain absolute product; however, some linalyl acetate will be lost, and a little residual wax and solvent still exist.

### SUMMARY OF THE INVENTION

The major purpose of the present invention is to provide a method and system for separating linalyl acetate from Lavender essential oil and preparing its derivatives, which could separate linalyl acetate from Lavender essential oil using supercritical fluid technology, and through esterification of acid resin catalyst, synthesize vitamin C derivative with anti-oxidizing oily fragrance for the cosmetics and pharmaceutical sectors.

To this end, the present invention provides a method of separating linalyl acetate from Lavender essential oil and preparing its derivatives, which applies the supercritical fluid technology to feed Lavender essential oil and supercritical solvent into the first separating tank, where linalyl acetate and linalool are separated from Lavender essential oil; then linalyl acetate is conveyed to the second separating tank, where linalyl acetate is separated with wax-containing oily substance to obtain high-concentration linalyl acetate; next linalool and vitamin C solution are conveyed to a reaction tank for esterification synthesis, and the reaction tank is filled with acid resin catalyst to obtain linalool-vitamin C derivative.

Besides, the present invention also provides a system of separating linalyl acetate from Lavender essential oil and preparing its derivatives, which comprises a first holding tank to accommodate Lavender extract; a first separating tank connected with the first holding tank, and used to separate linalyl acetate and linalool from Lavender essential oil; a supercritical fluid container connected with the first separating tank, and used to provide supercritical fluid; a second separating tank connected to the bottom of the first separating tank, and used to separate linalyl acetate with wax-containing oily substance; a second holding tank used to accommodate vitamin C solution; a reaction tank connected to the top of the first separating tank and the second holding tank, filled with acid resin catalyst for esterification synthesis of linalool and vitamin C solution to obtain linalool-vitamin C derivative; three electric heaters, separately set into the first, second separating tank and the reaction tank.

### BRIEF DESCRIPTION OF THE DRAWINGS

FIG. 1 is a sketch diagram of the continuous separation system of the preferred embodiment of the present invention.

### DETAILED DESCRIPTION OF THE INVENTION

The features and the advantages of the present invention will be more readily understood upon a thoughtful deliberation of the following detailed description of a preferred embodiment of the present invention with reference to the accompanying drawings:

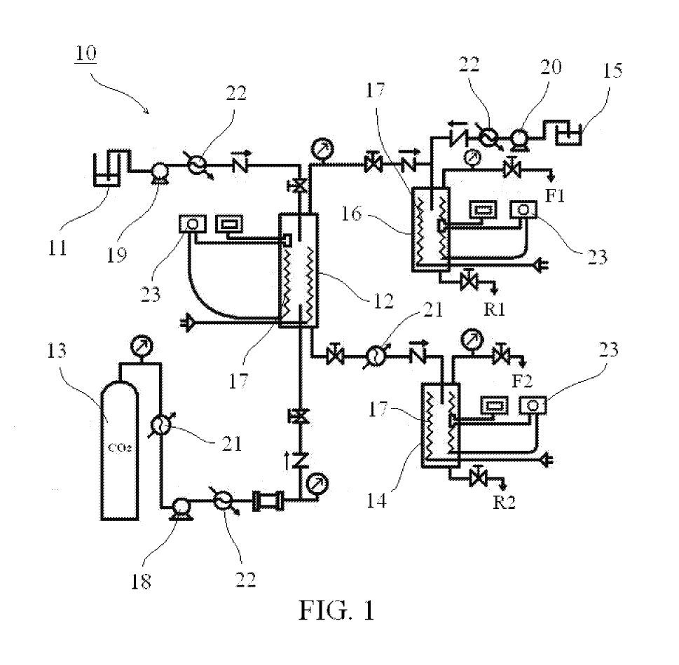

FIG. 1 depicts a preferred embodiment of the method and system for separating linalyl acetate from Lavender essential oil and preparing its derivatives with a supercritical fluid technology, wherein a system 10 is used to fabricate continuously linalyl acetate and its derivative; the system 10 comprises a first holding tank 11, a first separating tank 12, a supercritical fluid container 13, a second separating tank 14, a second holding tank 15, a reaction tank 16, three electric heaters 17, a high-pressure metering pump 18, a first metering pump 19, a second metering pump 20, two precoolers 21, three preheaters 22 and three temperature controllers 23, as well as valves.

The first holding tank 11 is used to accommodate Lavender essential oil, and the first separating tank 12 is connected with the first holding tank 11; made of a stainless steel tank and monomers, the separating tank could separate linalyl acetate and linalool from Lavender essential oil; the supercritical fluid container 13 is a steel cylinder containing CO₂ supercritical fluid, which is connected with the first separating tank 12 to provide CO₂ supercritical fluid; the second separating tank 14 is connected to the bottom of the first separating tank 12; made also of stainless steel and monomers, the separating tank could separate linalyl acetate with wax-containing oily substance; the second holding tank 15 is used to accommodate vitamin C solution, and the reaction tank 16 is connected to the top of the first separating tank 12 and the second holding tank 14; made of stainless steel tank, it is filled with acid resin catalyst, enabling esterification synthesis between linalool and vitamin C solution to obtain linalool-vitamin C derivative; the electric heaters 17 are separately set into the first/second separating tanks 12, 14 and the reaction tank 16; the high-pressure metering pump 18 is connected between the supercritical fluid container 13 and the first separating tank 12; the first metering pump 19 is connected between the first holding tank 11 and the first separating tank 12; the second metering pump 20 is connected between the second holding tank 15 and the first separating tank 12; the precoolers 21 are connected between the supercritical fluid container 13 and the high-pressure metering pump 18 as well as between the first and second separating tanks 12, 14; the preheaters 22 are separately connected between the high-pressure metering pump 18, metering pumps 19, 20 and the first/second separating tanks 12, 14; the temperature controllers 23 are separately connected with the electric heaters 17.

According to the method of separating linalyl acetate from Lavender essential oil and preparing its derivative, it is required to feed Lavender essential oil and supercritical solvent into the first separating tank 12 at a predefined rate, such that linalyl acetate and linalool can be separated from Lavender essential oil in the first separating tank 12; then linalyl acetate is conveyed to the second separating tank 14, where linalyl acetate is separated with wax-containing oily substance to obtain high-concentration linalyl acetate; next, linalool and vitamin C solution are conveyed to the reaction tank 16 for esterification synthesis; the reaction tank 16 is filled with acid resin catalyst to obtain linalool-vitamin C derivative. Namely, it is required to open the supercritical fluid container 13, control the pressure of the first separating tank 12 by the high-pressure metering pump 18 and valve, and control the temperature of the first separating tank 12 by the temperature controller 23; next open the first metering pump 19, control Lavender essential oil entering into the first separating tank 12 to separate high-concentration linalyl acetate and linalool; of which linalyl acetate is located at bottom of the first separating tank 12, and linalool located at top of the first separating tank 12.

Then, linalyl acetate at bottom of the first separating tank 12 enters into the second separating tank 14, the valve is used to control the pressure of the second separating tank 14, and the temperature controller 23 used to control the temperature of the second separating tank 14; thus, high-purity linalyl acetate is separated at top (F2) of the second separating tank 14, wax-containing oily substance in Lavender essential oil is collected at bottom (R2) in the second separating tank 14.

Next, open the second metering pump 20, such that 10% vitamin C solution in the second holding tank 15 is mixed with linalool at top of the first separating tank 12 and then fed into the reaction tank 16 for esterification, and valve is used to control the pressure of the reaction tank 16, and the temperature controller 23 is used to control the temperature of the reaction tank 16. After reaction, the sample of reactant-linalool-vitamin C derivative (linalyl ascorbate) is collected at top (F1) of the reaction tank 16, and remaining linalool, linalyl acetate and vitamin C are collected at bottom (R1) of the reaction tank 16.

Operating conditions of said first separating tank 12: temperature: 40-60° C., pressure: 8-9 MPa, flow rate of supercritical fluid: 3.0 L/hr, feed rate of Lavender essential oil: 0.5 L/hr. Operating conditions of said second separating tank 14: temperature: −5° C., pressure: 8 MPa. Operating conditions of said reaction tank 16: temperature: 100-120° C., pressure: 8-9 MPa, flow rate of supercritical fluid: 3.0 L/hr, and feed rate of vitamin C solution: 0.1 L/hr.

After Lavender essential oil is separated continuously by the first/second separating tanks 12, 14, samples (linalyl acetate, wax-containing oily substance) at top (F2) and bottom (R2) of the second separating tank 14 are collected. Linalool at top of the first separating tank 12 is mixed with vitamin C solution and esterified in the reaction tank 16, then samples (linalool-vitamin C derivative, remaining linalool, linalyl acetate and vitamin C) at top (F1) and bottom (R1) of the reaction tank 16 are collected. After experimental analysis, these samples are quantified to calculate the concentration of linalool, linalyl acetate and linalool-vitamin C derivatives.

Thus, the separation effect of linalyl acetate in Lavender essential oil, the esterification effect of linalool-vitamin C derivative and the influence of linalyl acetate and linalool on HRV (Hearth Rate Variability) are described below:

### A. Separation Effect of Linalyl Acetate:

Lavender essential oil containing 30% linalyl acetate and 36% linalool is separated in the first separating tank 12 of the system 10, then linalyl acetate and linalool are collected to analyze their component distribution. As listed in Table 1, under operating pressure of 8 Mpa and temperature of 40° C., the solubility of linalyl acetate and linalool in the supercritical fluid is low, leading to poor separation effect. Under temperature of 60° C. and operating pressure of 9 MPa, the solubility of linalyl acetate and linalool in the supercritical fluid could be raised once the temperature and pressure are increased simultaneously.

TABLE 1

| | Linalyl acetate | Linalool | Linalyl acetate/ |
|---|---|---|---|
| | (%) | (%) | linalool |
| Feed | 30.0 ± 0.6 | 36.0 ± 0.8 | 0.83 |

Separator 12 (Top layer)

| | | | |
|---|---|---|---|
| 8 MPa | | | |
| 40° C. | 5.1 ± 0.2 | 20.5 ± 0.6 | 0.25 |
| 50° C. | 8.7 ± 0.1 | 22.0 ± 0.3 | 0.40 |
| 60° C. | 9.9 ± 0.2 | 23.0 ± 1.3 | 0.43 |
| 9 MPa | | | |
| 40° C. | 10.8 ± 0.5 | 22.7 ± 1.0 | 0.47 |
| 50° C. | 12.3 ± 0.3 | 24.8 ± 1.2 | 0.50 |
| 60° C. | 12.8 ± 0.2 | 25.4 ± 0.8 | 0.52 |

Separator 14 (F2)

| | | | |
|---|---|---|---|
| 8 MPa | | | |
| 40° C. | 38.8 ± 2.5 | 32.5 ± 1.7 | 1.19 |
| 50° C. | 43.4 ± 2.6 | 35.9 ± 1.8 | 1.21 |
| 60° C. | 47.6 ± 0.9 | 38.3 ± 0.6 | 1.24 |
| 9 MPa | | | |
| 50° C. | 47.5 ± 2.1 | 36.4 ± 1.3 | 1.30 |
| 60° C. | 55.0 ± 1.5 | 39.0 ± 1.8 | 1.41 |

Under the operating conditions of −5° C., 8 MPa, wax in the essential oil containing paraffins could be mainly removed in the second separating tank 14. The results in Table 1 show that, under the conditions of 60° C., 9 MPa, linalyl acetate in the system 10 is increased to 55%, and linalool increased to 39%, with the ratio of linalyl acetate/linalool up to 1.41.

### B. Esterification Effect of Linalool-Vitamin C Derivative:

As listed in Table 2, under operating conditions of 120° C. and 8, 9 MPa in the reaction tank 16, the conversion rate is about 50-60%. Balanced reaction could occur due to generation of water, thus affecting the esterification synthesis efficiency. In the case of reaction, catalyst is used to fix anionic group (—SO₃⁻) onto the surface of polymer matrix; product could be obtained through conversion when the reactant is adsorbed onto the acid resin layer for esterification under phase equilibrium state. In the case of extremely low temperature, activated energy required for catalysis could not be met; in the case of high temperature, ideal conversion rate could be easily obtained.

TABLE 2

| Pressure | Temperature | Yield |
|---|---|---|
| (MPa) | (° C.) | (%) |
| 8 | 100 | 16.5 ± 1.5 |
| 8 | 110 | 48.5 ± 3.2 |
| 8 | 120 | 56.2 ± 2.8 |
| 9 | 100 | 24.6 ± 2.0 |
| 9 | 110 | 50.1 ± 4.5 |
| 9 | 120 | 55.4 ± 1.6 |

In the present invention, the water-soluble vitamin C is converted into oil-soluble or fat-soluble vitamin C, helping to resolve the shortcoming of water-soluble vitamin C that, it is easily washed off or dissolved by water or sweat when it is applied to cosmetics or exposed to light, heat or oxygen. Linalool is also capable of resisting anxiety and providing pleasant fragrance like linalyl acetate. Hence, vitamin C and linalool are synthesized into linalool-vitamin C derivative, which has fragrance and anti-anxiety aromatherapy effect, other than antioxidizing and skin whitening function of fat (oil)-soluble vitamin C.

### C. Influence of Linalyl Acetate and Linalool on HRV:

Next, Lavender essential oil and linalyl acetate at top (F2) of the second separating tank 14 are subject to essential oil inhalation test, by comparing the variation of HRV (Heart Rate Variability) before or after essential oil and F2 samples are used. The test was conducted from 08:30 to 12:30, in a 21.3 m² room with luminosity of 58 lux, indoor temperature of 24±1° C. and humidity of 60-70%. After the test samples of essential oil/water were mixed by a ratio of 1:75, they were placed in an ultrasonic sprayer to agitate into extract's misty molecule, and subject to 15 minutes inhalation with a spacing of 60 cm from the testee, and then 5 minutes HRV was measured immediately. The data analysis of HRV involved time domain and frequency domain analysis. Of which, time domain was used to measure the testee's MHR (Mean Heart Rate) and SDNN (standard deviation of all normal to normal intervals). Frequency domain was used to measure the testee's TP (total power), LF value (low frequency components) within 0.04-0.15 Hz, HF value (high frequency components) within 0.15-0.4 Hz, and the ratio of LF/HF was calculated. With the variation of the ratio of LF/HF, if HF is increased when the concentration of linalyl acetate is raised, the ratio of LF/HF declines, making people relaxed and comfortable. As listed in Table 3, 55% linalyl acetate at top (F2) of the second separating tank 14 and Lavender essential oil (30% linalyl acetate) increases by 72% and 41%, respectively, as compared with HF value of the control group (pure water group without adding essential oil). Thus, high-concentration linalyl acetate could be separated in the second separating tank 14, making it possible to improve parasympathetic functions and easily enable relaxation of the human body. Next, after inhalation test, MHR remains unchanged, but SDNN is increased slightly (about 7-8%), thus improving the circulation of heart without safety hazard.

TABLE 3

| | Items | | |
|---|---|---|---|
| | | Feed | Separator 14 (F2) |
| | | Linalyl acetate/ | Linalyl acetate/ |
| | Control | linalool | linalool |
| | (H₂O) | (30%/36%; 0.83) | (55%/39%; 1.41) |
| Time domain | | | |
| MHR (beats/min) | 72 ± 10 | 72 ± 12 | 74 ± 13 |
| SDNN (ms) | 54 ± 11 | 56 ± 15 | 58 ± 13 |
| Frequency domain | | | |
| TP (ms²) | 1435 ± 244 | 1701 ± 218 | 1908 ± 268 |
| LF (ms²) | 433 ± 127 | 554 ± 133 | 598 ± 130 |
| HF (ms²) | 403 ± 106 | 492 ± 129 | 695 ± 119 |
| LF/HF | 1.05 ± 0.18 | 1.13 ± 0.13 | 0.85 ± 0.16 |

In addition to increasing the concentration of linalyl acetate, Lavender essential oil could also be used to increase the concentration of those containing linalool and linalyl acetate from Citrus peel oil, and remove the off-flavor components in limonene that are vulnerable to light, temperature and oxygen. Meanwhile, low-temperature treatment in this system is conducted to remove wax containing paraffins in the Citrus peel oil.

Based on the method and system of the present invention for separating linalyl acetate from Lavender essential oil and preparing its derivatives, it is possible to apply supercritical fluid technology and continuous separation & reaction system to separate linalyl acetate from Lavender essential oil and prepare its derivatives; as compared with conventional steam distillation or solvent extraction method, the present invention could, by controlling the operating temperature and pressure, separate high-purity linalyl acetate, and prepare vitamin C derivative with anti-oxidizing oily fragrance for the cosmetics and pharmaceutical sectors; and, no residual solvent and toxicity occur in the separation and reaction process, enabling continuous operation of entire system.

What is claimed is:

1. A method of separating linalyl acetate from Lavender essential oil and preparing its derivatives, which applies the supercritical fluid technology to feed Lavender essential oil and supercritical solvent into the first separating tank, where linalyl acetate and linalool are separated from Lavender essential oil; then linalyl acetate is conveyed to the second separating tank, where linalyl acetate is separated with wax-containing oily substance to obtain high-concentration linalyl acetate; next linalool and vitamin C solution are conveyed to a reaction tank for esterification synthesis, and the reaction tank is filled with acid resin catalyst to obtain linalool-vitamin C derivative.

2. The method defined in claim 1, wherein said supercritical solvent is a CO₂ fluid in supercritical state.

3. The method defined in claim 1, wherein said first and second separating tanks are made of stainless steel tanks and filled with stainless steel monomers.

4. The method defined in claim 1, wherein Lavender essential oil is fed into the first separating tank at 0.5 L/hr, and supercritical solvent fed into the first separating tank at 3.0 L/hr under the operating conditions of 8-9 MPa and 40-60° C.

5. The method defined in claim 4, wherein linalyl acetate is separated at bottom of the first separating tank, and linalool separated at top of the first separating tank.

6. The method defined in claim 1, wherein linalyl acetate is conveyed to the second separating tank under the operating conditions of −5° C. and 8 MPa.

7. The method defined in claim 6, wherein linalyl acetate is separated at top of the second separating tank, and wax-containing oily substance separated at bottom of the second separating tank.

8. The method defined in claim 1, wherein said reaction tank is made of stainless steel tank and filled with acid resin catalyst.

9. The method defined in claim 1, wherein 10% vitamin C solution is fed into the reaction tank at 0.1 L/hr, and supercritical solvent fed into the reaction tank at 3.0 L/hr under the operating conditions of 100-120° C. and 8-9 MPa.

10. The method defined in claim 1, wherein comprises of:
a first holding tank, used to accommodate Lavender essential oil;
a first separating tank, connected with the first holding tank, and used to separate linalyl acetate and linalool from Lavender essential oil;
a supercritical fluid container, connected with the first separating tank, and used to provide supercritical fluid;
a second separating tank, connected to bottom of the first separating tank, and used to separate linalyl acetate with wax-containing oily substance;
a second holding tank, used to accommodate vitamin C solution;
a reaction tank, connected to top of the first separating tank and the second holding tank, filled with acid resin catalyst, enabling esterification synthesis of linalool and vitamin C solution to obtain linalool-vitamin C derivative; and
three electric heaters, separately set into the first, second separating tank and the reaction tank.

11. The system defined in claim 10, wherein the first and second separating tanks comprise a tank and a stainless steel monomer set in the tank; the electric heaters are set into the tank.

12. The method defined in claim 10, wherein the supercritical fluid container is used to contain CO₂ fluid in supercritical state.

13. The method defined in claim 10, wherein three temperature controllers thereof are connected separately with the electric heaters in the first, second separating tank and the reaction tank.

14. The method defined in claim 10, wherein it further comprising:
a high-pressure metering pump, connected between the supercritical fluid container and the first separating tank;
a first metering pump, connected between the first holding tank and the first separating tank;
a second metering pump, connected between the second holding tank and the first separating tank;
a precooler, connected between the supercritical fluid container and the high-pressure metering pump as well as between the first and second separating tanks;
three preheaters, connected separately between the high-pressure metering pump, reactant metering pump and the first and second separating tanks.
